(19) United States
(12) Patent Application Publication
(10) Pub. No.: US 2025/0295116 A1
(43) Pub. Date: Sep. 25, 2025

(54) METHOD FOR PRODUCING HYPOCHLOROUS ACID

(71) Applicant: YMK HOLDINGS LLC, Palm Beach, FL (US)

(72) Inventors: Bradley Gene DEROOS, Palm Beach, FL (US); Bryson Michael BEGLEY, Palm Beach, FL (US); Michael William CARPENTER, Palm Beach, FL (US)

(21) Appl. No.: 18/864,476

(22) PCT Filed: May 12, 2023
(86) PCT No.: PCT/US2023/022047

§ 371 Date: Nov. 8, 2024

Related U.S. Application Data
(60) Provisional application No. 63/341,214, filed on May 12, 2022.

(51) Int. Cl. A01N 59/00; C01B 11/04

(52) U.S. Cl. CPC A01N 59/00; C01B 11/04

(57) ABSTRACT

The present disclosure provides compositions, products, kits and methods for producing on demand hypochlorous acid solutions. The present disclosure provides a stable, dry mix product, kit or package that can be employed by an end-user to easily prepare a hypochlorous acid composition with desired features and functionality.

### CROSS-REFERENCE TO RELATED APPLICATIONS

This application claims the benefit of U.S. Provisional Application No. 63/341,214, filed May 12, 2022, which is incorporated by reference herein in its entirety.

### FIELD OF THE DISCLOSURE

The present application relates to the production of hypochlorous acid and compositions thereof, and to products and kits designed for an end-user to produce and use hypochlorous acid solutions.

### BACKGROUND

Hypochlorous acid is a non-toxic sanitizer, disinfectant, and decontaminant. It is naturally produced by the human body's immune system to fight infection. Neutrophils, a type of white blood cell, produce hypochlorous acid to kill bacteria, bacterial spores, viruses, and fungi.

While hypochlorous acid is produced efficiently and effectively by the human immune system, attempts at manufacturing commercially available hypochlorous acid have been disappointing, primarily because of the instability of hypochlorous acid, leading to a short shelf life. Because of this short shelf-life, there have been ongoing attempts to produce stable hypochlorous acid. Most attempts at manufacturing stable hypochlorous acid have used either (1) electrolysis of metal (e.g., sodium, calcium, lithium, potassium) chloride solutions, or (2) acidification of metal hypochlorite solutions (see, e.g., U.S. Published Appl. No. 2015/0119245). For example, attempts have been made to produce hypochlorous acid by the electrolysis of a brine (NaCl) solution. This is problematic, however, because of the specialized equipment required and cost associated with this manufacturing process.

Because of the many benefits associated with using hypochlorous acid solutions for sanitization, disinfection and decontamination, as well as due to the failure of others, there remains a long-felt and unmet need for a consumer-available hypochlorous acid products, kits or packages for onsite preparation of hypochlorous acid solutions, and a method for using the same. Moreover, to date there has been a need to create a product that allows a disinfectant solution (HOCl) that can be manufactured from dry mix components. The present disclosure provides a stable, dry mix product, kit or package that can be employed by an end-user to easily prepare a hypochlorous acid composition with desired features and functionality.

### BRIEF SUMMARY

The present disclosure provides products, kits and a process to create a disinfectant solution (HOCl) that is manufactured from dry mix components. The present disclosure provides products, kits and methods that have the following characteristics: large quantities of the hypochlorous acid solution can be rapidly generated on demand; the method of delivery can be selected as dictated by operational needs (e.g., fogger, sprayer, spray bottles); the products and kits are safe to use at the proposed concentration with no protective gear such as goggles, gloves, or aprons required; the dry mix chemicals have a long shelf life, and can be stored until needed; the cost for production is very low; the dry mix package size and configuration can be readily changed to accommodate end-user production needs; and shipping and logistics costs are low, since water can be used from on-site sources (e.g., tap water).

In a first aspect of the disclosure, a kit comprises a) a premeasured amount of a hypochlorite salt, for example, calcium hypochlorite, b) a premeasured mixture of a weak acid cation exchange resin and a strong acid cation exchange resin, and c) instructions for preparing hypochlorous acid, wherein components a) and b) are packaged individually.

In some aspects, upon following the instructions to prepare a hypochlorous acid solution, the resulting solution has a free available chlorine concentration between about 7000 and about 700 ppm. In some aspects, the resulting solution has a free available chlorine concentration between about 7000 and about 4000 ppm. Solutions with free available chlorine concentration levels between about 7000 and about 4000 ppm are useful for harder to kill microorganisms, and/or for applying the solution to surfaces where only a short contact time is possible. Suitable applications include treating surfaces that are possible contaminated with Bacillus anthracis (anthrax) or Clostridioides difficile (C. difficile). In some aspects,

In some aspects, upon following the instructions to prepare a hypochlorous acid solution, the resulting solution has a free available chlorine concentration between about 1000 and about 700 ppm. Hypochlorous acid solutions having a free available chlorine concentration between about 1000 and about 700 ppm are suitable for general surface decontamination or disinfection.

In some aspects, upon following the instructions to prepare a hypochlorous acid solution, the resulting solution has a free available chlorine concentration between about 700 ppm and about 50 ppm. In one aspect, the resulting solution has a free available chlorine concentration between about 200 and about 50 ppm. Solutions with free available chlorine concentration levels between about 200 and about 50 ppm are particularly suitable for treating mammalian skin or mammalian wounds, including wounds on humans.

In some aspects, the hypochlorous acid solution has a pH of about 4 to about 7.

In some aspects, the premeasured amount of calcium hypochlorite is in an amount to provide about 1.7 g/L to about 1.8 g/L for a predetermined amount of water.

In some aspects, the premeasured mixture of the weak acid cation exchange resin and the strong acid cation exchange resin is provided in an amount to provide 6 g/L to about 20 g/L of the weak acid cation exchange resin for a predetermined amount of water, and about 6 g/L to about 12 g/L of a strong acid cation exchange resin for a predetermined amount of water.

In some aspects, the premeasured mixture of the weak acid cation exchange resin and the strong acid cation exchange resin is provided in an amount equal to about 10 g of weak acid cation exchange resin/L of water based upon a predetermined amount of water; and about 8 g of the strong acid cation exchange resin/L based upon a predetermined amount of water.

In some aspects, the premeasured amount of calcium hypochlorite is in an amount of about 0.1 g/L to about 0.2 g/L for a predetermined amount of water.

In some aspects, the premeasured mixture of the weak acid cation exchange resin and the strong acid cation exchange resin is provided in an amount to provide 0.3 g/L to about 2.3 g/L of the weak acid cation exchange resin for a predetermined amount of water; and about 0.3 g/L to about 1.4 g/L of a strong acid cation exchange resin for a predetermined amount of water.

In some aspects, the premeasured mixture of the weak acid cation exchange resin and the strong acid cation exchange resin is provided in an amount equal to about 1.1 g of weak acid cation exchange resin/L of water based upon a predetermined amount of water; and about 0.9 g of the strong acid cation exchange resin/L based upon a predetermined amount of water.

In some aspects, the weak acid cation exchange resin is a hydrogen form of a weak acid cation exchange resin and comprises carboxylic acid functional groups and the strong acid cation exchange resin is a hydrogen form of a strong acid cation exchange resin and comprises sulfonic acid functional groups.

In some aspects, the kit further comprises d) a premeasured amount of a surfactant.

In some aspects, each of components a) and component b), and optionally component d), is packaged in an individual, air-tight and water-tight container.

In some aspects, the premeasured amount of the surfactant is an amount necessary to constitute 0.1% to 5% by weight of the hypochlorous acid solution once prepared.

In some aspects, the premeasured amount of the surfactant is an amount necessary to constitute 0.5% to 1% by weight of the hypochlorous acid solution once prepared.

In some aspects, the surfactant is selected from the group consisting of dioctyl sulfosuccinates, alkyldiphenyloxide disulfonates, polysorbates, ethoxylated polyoxypropylene block copolymer, alcohol ethoxylates, sodium polyacrylate polymer, sodium lauriminodipropionate, sodium octyliminodipropionate, and combinations thereof.

In some aspects, components a) and b), and optionally d), are packaged individually in 3-ply bags comprising oriented polypropylene, metalized polyethylene terephthalate, and linear low-density polyethylene.

In some aspects, the instructions for preparing a hypochlorous acid solution provide for admixing component a), component b), optionally component d), and a predetermined amount of water.

In some aspects, upon following the instructions included with the kit to prepare a hypochlorous acid solution, the prepared hypochlorous acid solution is further diluted with water.

In some aspects, the premeasured amounts of calcium hypochlorite and mixture of weak acid cation exchange resin and strong acid cation exchange resin are present in amounts effective to produce hypochlorite acid in an amount sufficient to reduce the concentration of a measured microorganism by 99%, for example 99.9% when remaining in contact with the surface for about 30 seconds to about 2 minutes. In certain aspects, the amounts of calcium hypochlorite and mixture of weak acid cation exchange resin and strong acid cation exchange resin are present in amounts effective to produce hypochlorite acid in an amount sufficient to kill 99.99% of bacteria on a surface. The products, kits and process of the disclosure premeasured amounts of calcium hypochlorite and mixture of weak acid cation exchange resin and strong acid cation exchange resin are present in amounts effective to produce hypochlorite acid in an amount sufficient to kill bacteria, viruses and fungi to a level of 99.9% of an original bacteria, virus or fungi count over a specified surface area of an inanimate object, plant or animal when remaining in contact with the surface for about 30 seconds to about 2 minutes. The ingredients can be provided in amounts sufficient to provide hypochlorous acid solutions capable of disinfecting surface areas from about 10 square feet to about 20,000 square feet.

In another aspect of the disclosure, hypochlorous acid is prepared by dissolving a premeasured amount of calcium hypochlorite in water, adding a premeasured amount of a mixture of a weak acid cation exchange resin and a strong acid cation exchange resin to water, and stirring, wherein the premeasured amounts of the calcium hypochlorite and the mixture and water are in effective amounts to produce a hypochlorous acid solution which has a pH of about 4 to about 7 when stirring is halted.

In some aspects, the free available chlorine concentration of the hypochlorous acid solution is between about 7000 ppm and about 50 ppm.

In some aspects, the free available chlorine concentration of the hypochlorous acid solution is between about 7000 ppm and about 4000 ppm.

In some aspects, the free available chlorine concentration of the hypochlorous acid solution is between about 4000 ppm and about 700 ppm.

In some aspects, the free available chlorine concentration of the hypochlorous acid solution is between about 1000 ppm and about 700 ppm.

In some aspects, the free available chlorine concentration of the hypochlorous acid solution is between about 1000 ppm and about 200 ppm.

In some aspects, the free available chlorine concentration of the hypochlorous acid solution is between about 200 ppm and about 50 ppm.

In some aspects, the free available chlorine concentration of the hypochlorous acid solution remains between about 1000 and 700 ppm when stored at room temperature for up to 48 hours.

In some aspects, the free available chlorine concentration of the hypochlorous acid solution remains between about 200 and 50 ppm when stored at room temperature for up to 48 hours.

In some aspects, the premeasured amount of calcium hypochlorite is in an amount of about 1.7 g/L to about 1.8 g/L for a predetermined amount of water.

In some aspects, the premeasured mixture of the weak acid cation exchange resin and the strong acid cation exchange resin is provided in an amount to provide 6 g/L to about 20 g/L of the weak acid cation exchange resin for a predetermined amount of water; and about 6 g/L to about 12 g/L of a strong acid cation exchange resin for a predetermined amount of water.

In some aspects, the premeasured mixture of the weak acid cation exchange resin and the strong acid cation exchange resin is provided in an amount equal to 10 g of weak acid cation exchange resin/L of water based upon a predetermined amount of water; and about 8 g of the strong acid cation exchange resin/L based upon a predetermined amount of water.

In some aspects, the premeasured amount of calcium hypochlorite is in an amount of about 0.1 g/L to about 0.2 g/L for a predetermined amount of water.

In some aspects, the premeasured mixture of the weak acid cation exchange resin and the strong acid cation exchange resin is provided in an amount to provide 0.3 g/L to about 2.3 g/L of the weak acid cation exchange resin for a predetermined amount of water; and about 0.3 g/L to about 1.4 g/L of a strong acid cation exchange resin for a predetermined amount of water.

In some aspects, the premeasured mixture of the weak acid cation exchange resin and the strong acid cation exchange resin is provided in an amount equal to about 1.1 g of weak acid cation exchange resin/L of water based upon a predetermined amount of water; and about 0.9 g of the strong acid cation exchange resin/L based upon a predetermined amount of water.

In some aspects, the process further comprising adding a premeasured amount of a surfactant with stirring.

In some aspects, the premeasured amount of the surfactant is an amount necessary to constitute 0.1% to 5% by weight of the hypochlorous acid solution once prepared.

In some aspects, the premeasured amount of the surfactant is an amount necessary to constitute 0.5% to 1% by weight of the hypochlorous acid solution once prepared.

In some aspects, the surfactant is selected from the group consisting of dioctyl sulfosuccinates, alkyldiphenyloxide disulfonates, polysorbates, ethoxylated polyoxypropylene block copolymer, alcohol ethoxylates, sodium polyacrylate polymer, sodium lauriminodipropionate, sodium octyliminodipropionate, and combinations thereof.

In some aspects, the solution is stirred for about 3 minutes to about 5 minutes.

In some aspects, the solution is stirred for about 4 minutes.

In some aspects, the weak acid cation exchange resin is a hydrogen form of a weak acid cation exchange resin and comprises carboxylic acid functional groups and the strong acid cation exchange resin is a hydrogen form of a strong acid cation exchange resin and comprises sulfonic acid functional groups.

In some aspects, the calcium hypochlorite and the mixture of weak acid cation exchange resin and strong acid cation exchange resin, and optionally a surfactant, are admixed by pouring from individual packages for a) calcium hypochlorite and b) the mixture of weak acid cation exchange resin and strong acid cation exchange resin.

In some aspects, the calcium hypochlorite is packaged in a first 3-ply bag, the mixture of weak acid cation exchange resin and strong acid cation exchange resin is packaged in a second 3-ply bag, and optionally a surfactant is packaged in a third 3-ply bag, and the calcium hypochlorite and mixture of resins, and optionally the surfactant, are admixed with a predetermined amount of water.

In some aspects, the hypochlorous acid solution is further diluted with water.

In some aspects, the premeasured amounts of calcium hypochlorite and mixture of weak acid cation exchange resin and strong acid cation exchange resin are present in amounts effective to produce hypochlorite acid in an amount sufficient to reduce the concentration of a measured microorganism by 99%, for example 99.9% when remaining in contact with the surface for about 30 seconds to about 2 minutes. In certain aspects, the amounts of calcium hypochlorite and mixture of weak acid cation exchange resin and strong acid cation exchange resin are present in amounts effective to produce hypochlorite acid in an amount sufficient to kill 99.99% of bacteria on a surface. The products, kits and process of the disclosure premeasured amounts of calcium hypochlorite and mixture of weak acid cation exchange resin and strong acid cation exchange resin are present in amounts effective to produce hypochlorite acid in an amount sufficient to kill bacteria, viruses and fungi to a level of 99.9% of an original bacteria, virus or fungi count over a specified surface area of an inanimate object, plant or animal when remaining in contact with the surface for about 30 seconds to about 2 minutes. The ingredients can be provided in amounts sufficient to provide hypochlorous acid solutions capable of disinfecting surface areas from about 10 square feet to about 20,000 square feet.

### DESCRIPTION OF FIGURES

FIG. 1 shows the concentration over time of a hypochlorous acid composition made according to the disclosure and stored at room temperature for 48 hours.

### DETAILED DESCRIPTION

The present disclosure concerns methods and compositions for producing on-demand hypochlorous acid solutions.

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those disclosed herein can be used in practice or testing of the present disclosure, suitable methods and materials are disclosed below. The materials, methods and examples are illustrative only and are not intended to be limiting. Other features and advantages of the disclosure will be apparent from the detailed description and from the claims.

### I. Definitions

In order to further understand this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Throughout this disclosure, various aspects are presented in a range format. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither, or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values, which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are disclosed herein with the language “comprising,” otherwise analogous aspects disclosed in terms of “consisting of” and/or “consisting essentially of” are also provided.

### II. Hypochlorite

In some aspects of the disclosure, a stable composition of hypochlorous acid can be produced using a hypochlorite as a starting material. In some aspects, the hypochlorite is sodium hypochlorite, calcium hypochlorite, lithium hypochlorite, or potassium hypochlorite. In some aspects, the hypochlorite is calcium hypochlorite.

Calcium hypochlorite has the molecular formula of Ca(ClO)₂ and a molecular weight of 142.974 g/mol. Its CAS number is 7778-54-3. Its specific gravity is 1.21 g/cm³.

Calcium hypochlorite is commercially available. Methods for producing calcium hypochlorite are well known in the art. For example, calcium hypochlorite may be produced by reacting calcium hydroxide with chlorine gas in the following reaction:

2Cl₂+2Ca(OH)₂→Ca(OCl)₂+CaCl₂+2H₂O.

### III. Strongly and Weakly Acidic Cation Exchange Resins

Ion exchange resins are composed of polymer matrices to which ionic “functional groups” of either positively charged ions (cations) or negatively charged ions (anions) are permanently bound. Some specialty resins have both types of functionality. These functional groups have a net negative or positive charge that allows them to readily attract counter ions, or ions of an opposing charge. As a liquid stream flows through the ion exchange resin, the counter ions can be replaced by ions of a similar charge.

Broadly speaking, resins are named for the type of ions they exchange, meaning that cationic resins exchange positively charged ions, while anionic resins exchange negatively charged ions. Type of ion exchange resins include strong acid cation resins, weak acid cation resins, strong base anion resins, weak base anion resins, and specialty resins.

Strongly acidic cation (SAC) exchange resins are composed of a polymer matrix to which anionic functional groups are bound, such as sulphonate (SO³⁻). The resins typically derive their exchange activity from sulfonic functional groups. Strongly acidic cation resins are available commercially from a number of sources. Mitsubishi offers DIAION series of SAC resins. Millipore offers AMBERLITE, DOWEX 50 and AMERLYST series of SAC resins. Purolite and ResinTech also provide SAC resins. In some aspects, the SAC exchange resin is ResinTech CG10-H.

Weakly acidic cation (WAC) exchange resins are composed of a polymer matrix to which carboxylic functional groups are bound, such as carboxylic acid (COOH). Weakly acidic cation resins are available commercially from a number of sources. Mitsubishi offers DIAION series of SAC resins. DuPont offers AmberLite MAC-3. Purolite and ResinTech also provide WAC resins. In some aspects, the WAC exchange resin is ResinTech WACG-HP. In some aspects, the WAC exchange resin is ResinTech WACMP. In some aspects, the WAC exchange resin is AmberLite MAC-3.

### IV. Method for Producing Hypochlorous Acid

Certain aspects of the disclosure are directed to a process for producing hypochlorous acid.

The production principle for making a hypochlorous acid (HOCl) solution from the dry mix ingredients of the disclosure is straightforward-the pH of a calcium hypochlorite bleach solution is lowered from 11.0 to 7.0 to create HOCl via ion exchange. Weak acid cation (WAC) exchange resin and strong acid cation (SAC) exchange resins are polymer resins in which hydrogen ions are bound within the polymer network. The hydrogen ions are released from the polymer network when cations for which the resin has greater affinity reach an ion exchange site. In the case of the dry mix disinfection system of the disclosure, calcium hypochlorite—with the chemical formula Ca(OCl)₂—is mixed with water to create a calcium hypochlorite bleach solution with a pH of approximately 11. The concentration is controlled via the ratio of calcium hypochlorite to water used. To make HOCl, WAC and SAC exchange resins are mixed into the calcium hypochlorite solution at carefully specified mix ratios. At this point, ion exchange occurs wherein the calcium ions are exchanged with the hydrogen ions bound to the resin. This results in a transformation of the solution from calcium hypochlorite bleach to hypochlorous acid. This transformation occurs rapidly as the weak acid cation exchange resin has much higher affinity for calcium ions than it does for hydrogen ions. The use of a combination of WAC exchange resin and SAC exchange resin provides for a controlled reaction system that reliably produces a reaction solution and a hypoochlorous acid solution at an appropriate pH range, and having sufficient stability and safety for end-user production and use. The ion exchange process serves to create a solution that has a pH of between 4 and 7.5.

The production methods are scalable in nature; the technique can be used to generate HOCl in quantities of quarts to hundreds of gallons. This HOCl manufacturing techniques allows large quantities of HOCl to be safely produced at a low cost.

In certain aspects of the disclosure, a premeasured amount of calcium hypochlorite is dissolved in a predetermined amount of water to produce a calcium hypochlorite solution. In some aspects of the disclosure, a premeasured amount of calcium hypochlorite is about 0.05 g to about 10 g. In some aspects, the premeasured amount of calcium hypochlorite is about 0.1 g to about 2.3 g. In some aspects, the premeasured amount of calcium hypochlorite is about 0.05 g to about 0.5 g. In some aspects, the premeasured amount of calcium hypochlorite is about 0.1 g to about 0.2 g. In some aspects, the premeasured amount of calcium hypochlorite is about 1.5 g to about 2 g. In some aspects, the premeasured amount of calcium hypochlorite is about 1.7 g to about 1.8 g. In some aspects, the premeasured amount of calcium hypochlorite is about 0.25 g to about 0.75 g. In some aspects, the premeasured amount of calcium hypochlorite is about 0.4 g to about 0.5 g. In some aspects, the premeasured amount of calcium hypochlorite is about 6 g to about 8 g. In some aspects, the premeasured amount of calcium hypochlorite is about 6.8 g to about 7.2 g.

In certain aspects of the disclosure, a premeasured amount of calcium hypochlorite is dissolved in water to produce a calcium hypochlorite solution with a concentration of 0.05 g/L to about 10 g/L. In some aspects, the premeasured amount of calcium hypochlorite is dissolved in water to produce a calcium hypochlorite solution with a concentration of about 0.1 g/L to about 2.3 g/L. In some aspects, the premeasured amount of calcium hypochlorite is dissolved in water to produce a calcium hypochlorite solution with a concentration of about 0.05 g/L to about 0.5 g/L. In some aspects, the premeasured amount of calcium hypochlorite is dissolved in water to produce a calcium hypochlorite solution with a concentration of about 0.1 g/L to about 0.2 g/L. In some aspects, the premeasured amount of calcium hypochlorite is dissolved in water to produce a calcium hypochlorite solution with a concentration of about 1.5 g/L to about 2 g/L. In some aspects, the premeasured amount of calcium hypochlorite is dissolved in water to produce a calcium hypochlorite solution with a concentration of about 1.7 g/L to about 1.8 g/L.

The calcium hypochlorite solution is then acidified to produce hypochlorous acid. The calcium hypochlorite solution is acidified by adding a premeasured amount of one or more cation exchange resins to the calcium hypochlorite solution. In some aspects, the calcium hypochlorite solution is acidified by adding a mixture of a strongly acidic cation exchange resin (SAC exchange resin) and a weakly acidic cation exchange resin (WAC exchange resin). In some aspects, the SAC exchange resin is a hydrogen form SAC exchange resin. In some aspects, the WAC exchange resin is a hydrogen form WAC exchange resin. One skilled in the art would understand that the hydrogen SAC exchange resin is manufactured in a hydrated state and can possibly dehydrate over time. As explained further below, the weight of resin to be added to the calcium hypochlorite solution is calculated prior to its addition. If the hydrogen SAC resin and/or WAC resin is allowed to excessively dehydrate over time, one skilled in the art would appreciate the increased risk of adding an overage of resin to the calcium hypochlorite solution, which may result in the excessive generation of chlorine gas. Thus, unless otherwise specified, the hydrogen SAC resin used in the disclosed processes is in a substantially fully hydrated state.

In certain aspects of the disclosure, a premeasured mixture of WAC exchange resin and SAC exchange resin is added to the calcium hypochlorite solution to provide a WAC exchange resin concentration of about 0.3 g/L to about 20 g/L and a SAC exchange resin concentration of about 0.3 g/L to about 12 g/L. In some aspects, the premeasured mixture of WAC exchange resin and SAC exchange resin is added to the calcium hypochlorite solution to provide a WAC exchange resin concentration of about 0.3 g/L to about 2.3 g/L and a SAC exchange resin concentration of about 0.3 g/L to about 1.4 g/L. In some aspects, the premeasured mixture of WAC exchange resin and SAC exchange resin is added to the calcium hypochlorite solution to provide a WAC exchange resin concentration of about 6 g/L to about 20 g/L and a SAC exchange resin concentration of about 6 g/L to about 12 g/L. In some aspects, the premeasured mixture of WAC exchange resin and SAC exchange resin is added to the calcium hypochlorite solution to provide a WAC exchange resin concentration of about 8 g/L to about 12 g/L and a SAC exchange resin concentration of about 8 g/L to about 10 g/L. In some aspects, the premeasured mixture of WAC exchange resin and SAC exchange resin is added to the calcium hypochlorite solution to provide a WAC exchange resin concentration of about 10 g/L and a SAC exchange resin concentration of about 8 g/L. In some aspects, the premeasured mixture of WAC exchange resin and SAC exchange resin is added to the calcium hypochlorite solution to provide a WAC exchange resin concentration of about 1.1 g/L and a SAC exchange resin concentration of about 0.9 g/L.

The process of preparing a hypochlorous acid solution of a predetermined strength and volume can be practiced by resort to products and kits of the disclosure. The individual packets of (a) calcium hypochlorite, (b) mixture of the WAC exchange resin, SAC exchange resin, and (c) optionally surfactant are opened and poured into a container. Water is also added to the container. The resulting aqueous mixture of WAC exchange resin, SAC exchange resin, optionally surfactant, and calcium hypochlorite solution mixture is stirred for about 2 minutes to 10 minutes. In some aspects, the WAC exchange resin, SAC exchange resin, optionally surfactant, and calcium hypochlorite solution mixture is stirred for about 3 minutes to 5 minutes. In some aspects, the WAC exchange resin, SAC exchange resin, optionally surfactant, and calcium hypochlorite solution mixture is stirred for about 4 minutes.

In certain aspects of the disclosure, the WAC and SAC exchange resins, and optionally a surfactant, is added to the calcium hypochlorite solution and stirred in a non-reactive container. In some aspects, the mixture is stirred with a non-reactive stirring implement, such as a wooden paddle or a glass stirring rod. In certain aspects of the disclosure, after acidification, the resin is allowed to settle to the bottom of the container and the hypochlorous acid solution is poured off, preferably within about 2 to about 3 minutes of hypochlorous acid formation. In certain aspects of the disclosure, the hypochlorous acid solution is passed through a filter to ensure no resin is contained within the hypochlorous acid solution. In some aspects, the hypochlorous acid is filtered through a 350-micron filter (45-mesh size) or a 250-micron (60 mesh size) filter. It should be understood that the filter should be an appropriate size to prevent passage of the ion exchange resin.

In certain aspects of the disclosure, the hypochlorous acid can be bottled in a non-reactive container and capped with a non-reactive cap and liner or capped with a non-reactive sprayer and liner or capped with any other application mechanism that is non-reactive with hypochlorous acid. In some aspects, the hypochlorous acid can be stored in a non-reactive trigger spray bottle. In one embodiment, the spray bottle is opaque.

### V. Surfactants

In certain aspects of the disclosure, a premeasured amount of surfactant is added to the mixture of calcium hypochlorite solution and SAC and WAC exchange resins. In some aspects, the surfactant is dioctyl sulfosuccinate (for example, Polywet™ manufactured by PolyventivePlus], alkyldiphenyloxide disulfonates (for example, Dowfax™ manufactured by Dow), polysorbates (for example, polysorbate 20 or polysorbate 80), ethoxylated polyoxypropylene block copolymer (for example, Antarox® manufactured by Solvay], alcohol ethoxlates (for example, Rhodasurf® manufactured by Solvay), sodium polyacrylate polymer (for example, Flexisperse™ manufactured by Innovative Chemical Technologies, Inc.), sodium lauriminodipropionate (for example, Flexisurf™ LDP manufactured by Innovative Chemical Technologies, Inc.), sodium octyliminodipropionate (for example, Flexisurf™ EHDP manufactured by Innovative Chemical Technologies, Inc.), and combinations thereof.

In certain aspects of the disclosure, a premeasured amount of surfactant is added to the mixture of calcium hypochlorite solution and SAC and WAC exchange resins in an amount to constitute about 0.1% to about 5% by weight of the final hypochlorous acid solution. In some aspects, the surfactant is added in an amount to constitute about 0.5% to about 5% by weight of the final hypochlorous acid solution. In some aspects, the surfactant is added in an amount to constitute about 0.5% to about 2.5% by weight of the final hypochlorous acid solution. In some aspects, the surfactant is added in an amount to constitute about 0.5% to about 1% by weight of the final hypochlorous acid solution.

### VI. Kits and Products of the Disclosure

In certain aspects of the disclosure, the premeasured calcium hypochlorite, the premeasured mixture of WAC exchange resin and SAC exchange resin, and optionally the premeasured surfactant, are packaged individually in a kit. In some aspects, the premeasured amounts of calcium hypochlorite and mixture of weak acid cation exchange resin and strong acid cation exchange resin are present in amounts effective to produce hypochlorous acid in an amount sufficient to reduce the concentration of a measured microorganism by 99%, for example 99.9% when remaining in contact with the surface for about 30 seconds to about 2 minutes. In certain aspects, the amounts of calcium hypochlorite and mixture of weak acid cation exchange resin and strong acid cation exchange resin are present in amounts effective to produce hypochlorite acid in an amount sufficient to kill 99.99% of bacteria on a surface. The products, kits and process of the disclosure premeasured amounts of calcium hypochlorite and mixture of weak acid cation exchange resin and strong acid cation exchange resin are present in amounts effective to produce hypochlorite acid in an amount sufficient to kill bacteria, viruses and fungi to a level of 99.9% of an original bacteria, virus or fungi count over a specified surface area of an inanimate object, plant or animal when remaining in contact with the surface for about 30 seconds to about 2 minutes. The ingredients can be provided in amounts sufficient to provide hypochlorous acid solutions capable of disinfecting surface areas from about 10 square feet to about 20,000 square feet. In some aspects, the premeasured calcium hypochlorite, the premeasured WAC exchange resin and the premeasured SAC exchange resin, and optionally the premeasured surfactant are packaged individually to prevent degradation by moisture, heat, and/or light. The WAC and SAC can be prepackaged together as a mixture. In some aspects, the premeasured calcium hypochlorite, the premeasured WAC exchange resin, the premeasured SAC exchange resin (or premeasured mixture of WAC and SAC exchange resins), and optionally the premeasured surfactant are packaged individually in 3-ply bags comprising oriented polypropylene, metalized polyethylene terephthalate, and linear low-density polyethylene (for example, barrier pouches manufactured by Uline).

In certain aspects of the disclosure, the kit contains instructions for preparing hypochlorous acid from the kit components. In some aspects, the instructions are printed on a separate piece of paper in the kit, the instructions are printed on the packaging of the individual components in the kit, or the instructions are printed on the kit.

In certain aspects of the disclosure, a premeasured amount of calcium hypochlorite is packaged individually to prevent degradation by moisture, heat, and/or light. In some aspects of the disclosure, a premeasured amount of calcium hypochlorite is about 0.05 g to about 10 g is packaged individually. In some aspects, the premeasured amount of calcium hypochlorite is about 0.1 g to about 2.3 g is packaged individually. In some aspects, the premeasured amount of calcium hypochlorite is about 0.05 g to about 0.5 g is packaged individually. In some aspects, the premeasured amount of calcium hypochlorite is about 0.1 g to about 0.2 g is packaged individually. In some aspects, the premeasured amount of calcium hypochlorite is about 1.5 g to about 2 g is packaged individually. In some aspects, the premeasured amount of calcium hypochlorite is about 1.7 g to about 1.8 g is packaged individually. In some aspects, the premeasured amount of calcium hypochlorite is about 0.25 g to about 0.75 g is packaged individually. In some aspects, the premeasured amount of calcium hypochlorite is about 0.4 g to about 0.5 g is packaged individually. In some aspects, the premeasured amount of calcium hypochlorite is about 6 g to about 8 g is packaged individually. In some aspects, the premeasured amount of calcium hypochlorite is about 6.8 g to about 7.2 g is packaged individually.

In certain aspects of the disclosure, the premeasured amount of calcium hypochlorite is individually packaged in an amount sufficient to produce a calcium hypochlorite solution with a concentration of 0.05 g/L to about 10 g/L when dissolved in water. In some aspects, the premeasured amount of calcium hypochlorite is individually packaged in an amount sufficient to produce a calcium hypochlorite solution with a concentration of about 0.1 g/L to about 2.3 g/L when dissolved in water. In some aspects, the premeasured amount of calcium hypochlorite is individually packaged in an amount sufficient to produce a calcium hypochlorite solution with a concentration of about 0.05 g/L to about 0.5 g/L when dissolved in water. In some aspects, the premeasured amount of calcium hypochlorite is individually packaged in an amount sufficient to produce a calcium hypochlorite solution with a concentration of about 0.1 g/L to about 0.2 g/L when dissolved in water. In some aspects, the premeasured amount of calcium hypochlorite is individually packaged in an amount sufficient to produce a calcium hypochlorite solution with a concentration of about 1.5 g/L to about 2 g/L when dissolved in water. In some aspects, the premeasured amount of calcium hypochlorite is individually packaged in an amount sufficient to produce a calcium hypochlorite solution with a concentration of about 1.7 g/L to about 1.8 g/L when dissolved in water.

In certain aspects of the disclosure, the premeasured amounts of WAC exchange resin and SAC exchange resin are packaged individually (or packaged together as a mixture) to prevent degradation by moisture, heat, and/or light. In some aspects, the premeasured amounts of WAC exchange resin and SAC exchange resin are in an amount sufficient to provide a WAC exchange resin concentration of about 0.3 g/L to about 20 g/L and a SAC exchange resin concentration of about 0.3 g/L to about 12 g/L when added to the calcium hypochlorite solution. In some aspects, the premeasured amounts of WAC exchange resin and SAC exchange resin are in an amount sufficient to provide a WAC exchange resin concentration of about 0.3 g/L to about 2.3 g/L and a SAC exchange resin concentration of about 0.3 g/L to about 1.4 g/L when added to the calcium hypochlorite solution. In some aspects, the premeasured amounts of WAC exchange resin and SAC exchange resin are in an amount sufficient to provide a WAC exchange resin concentration of about 6 g/L to about 20 g/L and a SAC exchange resin concentration of about 6 g/L to about 12 g/L when added to the calcium hypochlorite solution. In some aspects, the premeasured amounts of WAC exchange resin and SAC exchange resin are in an amount sufficient to provide a WAC exchange resin concentration of about 8 g/L to about 12 g/L and a SAC exchange resin concentration of about 8 g/L to about 10 g/L when added to the calcium hypochlorite solution. In some aspects, the premeasured amounts of WAC exchange resin and SAC exchange resin are in an amount sufficient to provide a WAC exchange resin concentration of about 10 g/L and a SAC exchange resin concentration of about 8 g/L when added to the calcium hypochlorite solution. In some aspects, the premeasured amounts of WAC exchange resin and SAC exchange resin are in an amount sufficient to provide a WAC exchange resin concentration of about 1.1 g/L and a SAC exchange resin concentration of about 0.9 g/L when added to the calcium hypochlorite solution.

In certain aspects of the disclosure, a premeasured amount of surfactant is packaged individually and added to the mixture of calcium hypochlorite solution and SAC and WAC exchange resins in an amount to constitute about 0.1% to about 5% by weight of the final hypochlorous acid solution. In some aspects, the surfactant is added in an amount to constitute about 0.5% to about 5% by weight of the final hypochlorous acid solution. In some aspects, the surfactant is added in an amount to constitute about 0.5% to about 2.5% by weight of the final hypochlorous acid solution. In some aspects, the surfactant is added in an amount to constitute about 0.5% to about 1% by weight of the final hypochlorous acid solution.

### VII. Hypochlorous Acid

In certain aspects of the disclosure, the premeasured amounts of calcium hypochlorite and mixture of weak acid cation exchange resin and strong acid cation exchange resin are present in amounts effective to produce hypochlorous acid in an amount sufficient to reduce the concentration of a measured microorganism by 99%, for example 99.9% when remaining in contact with the surface for about 30 seconds to about 2 minutes. In certain aspects, the amounts of calcium hypochlorite and mixture of weak acid cation exchange resin and strong acid cation exchange resin are present in amounts effective to produce hypochlorite acid in an amount sufficient to kill 99.99% of bacteria on a surface. The products, kits and process of the disclosure premeasured amounts of calcium hypochlorite and mixture of weak acid cation exchange resin and strong acid cation exchange resin are present in amounts effective to produce hypochlorite acid in an amount sufficient to kill bacteria, viruses and fungi to a level of 99.9% of an original bacteria, virus or fungi count over a specified surface area of an inanimate object, plant or animal when remaining in contact with the surface for about 30 seconds to about 2 minutes. In some aspects, the hypochlorous acid produced according to the disclosure has a free available chlorine (FAC) concentration of about 50 to about 7000 ppm. In some aspects, the hypochlorous acid produced according to the disclosure has a FAC concentration of about 50 to 200 ppm. In some aspects, the hypochlorous acid produced according to the disclosure has a FAC concentration of about 50 to about 100 ppm. In some aspects, the hypochlorous acid produced according to the disclosure has a FAC concentration of about 50 to about 1500 ppm, or about 500 to about 1200 ppm. In some aspects, the hypochlorous acid produced according to the disclosure has a FAC concentration of about 200 to about 1100 ppm, or about 700 to about 1100 ppm. In some aspects, the hypochlorous acid produced according to the disclosure has a FAC concentration of about 700 to about 1000 ppm.

In certain aspects of the disclosure, the hypochlorous acid produced according to the disclosure has a pH of about 3 to about 8. In some aspects, the hypochlorous acid has a pH of about 4 to 7.

In certain aspects of the disclosure, the hypochlorous acid produced according to the disclosure may be further diluted to a preferred FAC concentration. A person of ordinary skill in the art would understand how to determine the necessary amount of water to be added to the hypochlorous acid solution to dilute it to a predetermined FAC concentration.

In certain aspects of the disclosure, the FAC concentration of a hypochlorous acid solution made according to the disclosure does not decrease by more than 50%, by more than 40%, by more than 30%, by more than 20%, or by more than 10% when stored at room temperature for 48 hours. In some aspects, the FAC concentration the hypochlorous acid does not decrease by more than 30%, by more than 20%, by more than 10%, or by more than 5% when stored at room temperature for 24 hours.

### VIII. Hypochlorous Acid Uses

Hypochlorous acid solution has a wide range of uses, including sanitization, disinfection, and decontamination. Examples of such uses include sanitizing and disinfecting in hospitals, nursing homes, long-term care facilities, emergency care facilities, ambulances, medical laboratories, doctors' offices, clinics, dental facilities, and other healthcare environments; daycare facilities and schools; hospitality industry settings such as restaurants, hotels, and cruise ships; retail food settings such as grocery stores and convenience stores; agricultural settings including livestock barns, veterinary facilities, and plant and seed treatment sites; gyms and workout facilities; building offices; general home use; and restrooms. Additionally, hypochlorous acid solutions can be applied or sprayed directly onto plants (leaves, stems, flowers and fruits), for example, in the floral industry to improve the shelf life of flowers. The hypochlorous acid solutions can also be applied or sprayed directly onto animals, for example for skin sanitization; wound care; and burn treatment.

Hypochlorous acid is known to effectively kill a wide variety of microorganisms with varying contact times (i.e., the amount of time the hypochlorous acid is in contact with the microorganism). Hypochlorous acid solution produced according to the disclosure was tested and found effective against the following microorganisms:

TABLE 1

| Bacteria: Broad-Spectrum & Hospital | Contact Time | |
|---|---|---|
| Staphylococcus aureus (ATCC 6538) | 1 | min |
| Pseudomonas aeruginosa (ATCC 15442) | 1 | min |
| Viruses: | | |
| Rhinovirus Type 37 (ATCC VR-1147, Strain 151-1) | 30 | sec |
| Human Coronavirus (ATCC VR-VR740, Strain 229E) | 30 | sec |

### EXAMPLES

### Example 1

Process for producing hypochlorous acid.

Hypochlorous acid was produced according to the following process:
1. Pour 1 L of water into non-reactive mixing container.
2. Add 1.7 g of calcium hypochlorite to mixing container and stir with a wooden paddle for approximately 30 seconds.
3. Add a premeasured mixture of 10 g of WAC exchange resin and 8 g of SAC exchange resin to the mixing container. Immediately begin stirring with the wooden paddle and stir for 4 minutes.
4. Allow resins to settle to bottom of mixing container before pouring hypochlorous acid solution into a second, non-reactive container for use. Hypochlorous acid solution may be filtered through a suitable filter to remove resin, as well.

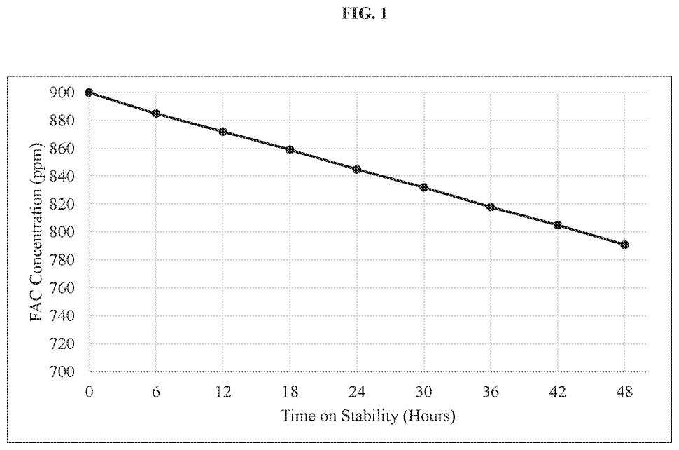

The hypochlorous acid was stored in a non-reactive container at room temperature and the FAC concentration was monitored over time. As shown in FIG. 1 and Table 2, below, the FAC concentration decreases by about 12% to from 900 ppm to 791 ppm when stored for 48 hours at room temperature.

TABLE 2

| Time on | FAC |
|---|---|
| Stability | Concentration |
| (hours) | (ppm) |
| 0 | 900 |
| 6 | 885 |
| 12 | 872 |
| 18 | 859 |
| 24 | 845 |
| 30 | 832 |
| 36 | 818 |
| 42 | 805 |
| 48 | 791 |

### Example 2

Preparing a Hypochlorous Acid Solution from a Kit

A hypochlorous acid solution is prepared using a method comprising the following steps that are similar to Example 1, except that the solid ingredients (1. calcium hypochlorite, 2. ion exchange resins, and 3. surfactant are prepackaged in individual, 3-ply barrier pouches manufactured by Uline. Step 1 involves the mixing of prepackaged ingredients and adding all the ingredients to a container, along with a predetermined volume of water. The primary purpose of this step is to prepare a solution of hypochlorous acid via reaction of WAC exchange resin and SAC exchange resin with calcium hypochlorite, which is achieved through good stirring so all the ingredients are properly dissolved. A surfactant can be optionally added during this step. The surfactant will aid in wetting surfaces that are hydrophobic. Step 2 involves decanting or otherwise removing the WAC exchange resin and SAC exchange resin which does not dissolve during the formation of hypochlorous acid solution.

### Example 3

Testing Hypochlorous Acid Solution from a Kit

A film of bacterial cells, or a film of viruses dried, on a surface of glass carriers was exposed to the test substance for specified exposure times or thirty seconds, or one minute. The test substance was a solution of Example 1 that was sprayed via a trigger spray bottle, four sprays at a distance of 6-8 inches from the carrier surface. Following exposure, the carriers were transferred to vessels containing neutralizing subculture medium. The subcultures were incubated and assayed for survivors. Appropriate culture purity, viability, organic soil load sterility, neutralizing subculture medium sterility, carrier sterility, carrier population and neutralization confirmation controls were performed. The results of the tests are reported in Table 1, above.

### OTHER ASPECTS

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific aspects thereof, it will be understood that the invention is capable of further modifications and this application is intended to cover any variations, uses, or adaptations of the invention following, in general, the principles of the invention and including such departures from the present disclosure that come within known or customary practice within the art to which the invention pertains and can be applied to the essential features hereinbefore set forth, and follows in the scope of the claimed invention.

What is claimed is:

1. A kit comprising a) a premeasured amount of calcium hypochlorite, b) a premeasured amount of a weak acid cation exchange resin and a premeasured amount of a strong acid cation exchange resin, and c) instructions for preparing hypochlorous acid, wherein component a) is packaged separately components b); and the weak acid cation exchange resin and the strong acid cation exchange resin can be packaged together as a mixture, or can be packaged separately from one another.

2. The kit according to claim 1, wherein upon following the instructions to prepare a hypochlorous acid solution, the resulting solution has a free available chlorine concentration between about 7000 ppm and about 50 ppm.

3. The kit according to claim 1, wherein upon following the instructions to prepare a hypochlorous acid solution, the resulting solution has a free available chlorine concentration between about 1000 and 700 ppm.

4. The kit according to claim 1, wherein upon following the instructions to prepare a hypochlorous acid solution, the resulting solution has a free available chlorine concentration between about 1000 and 200 ppm.

5. The kit according to claim 1, wherein upon following the instructions to prepare a hypochlorous acid solution, the resulting solution has a free available chlorine concentration between about 7000 and 4000 ppm.

6. The kit according to claim 5, wherein upon following the instructions to prepare a hypochlorous acid solution, the resulting solution has a free available chlorine concentration between about 200 and 50 ppm.

7. The kit according to any of claims 1-6, wherein the hypochlorous acid solution has a pH of about 4 to about 7.

8. The kit according to claim 3, wherein the premeasured amount of calcium hypochlorite is provided in an amount to provide about 1.7 g/L to about 1.8 g/L for a predetermined amount of water.

9. The kit according to claim 8, wherein the premeasured mixture of the weak acid cation exchange resin and the strong acid cation exchange resin is provided in an amount to provide about 6 g/L to about 20 g/L of the weak acid cation exchange resin for a predetermined amount of water; and about 6 g/L to about 12 g/L of a strong acid cation exchange resin for a predetermined amount of water.

10. The kit according to any one of claim 9, wherein the premeasured mixture of the weak acid cation exchange resin and the strong acid cation exchange resin is provided in an amount equal to about 10 g of weak acid cation exchange resin/L of water based upon a predetermined amount of water; and about 8 g of the strong acid cation exchange resin/L based upon a predetermined amount of water.

11. The kit according to claim 6, wherein the premeasured amount of calcium hypochlorite is in an amount of about 0.1 g/L to about 0.2 g/L for a predetermined amount of water.

12. The kit according to claim 11, wherein the premeasured mixture of the weak acid cation exchange resin and the strong acid cation exchange resin is provided in an amount to provide 0.3 g/L to about 2.3 g/L of the weak acid cation exchange resin for a predetermined amount of water; and about 0.3 g/L to about 1.4 g/L of a strong acid cation exchange resin for a predetermined amount of water.

13. The kit according to claim 12, wherein the premeasured mixture of the weak acid cation exchange resin and the strong acid cation exchange resin is provided in an amount equal to about 1.1 g of weak acid cation exchange resin/L of water based upon a predetermined amount of water; and about 0.9 g of the strong acid cation exchange resin/L based upon a predetermined amount of water.

14. The kit according to any one of claims 1-13, wherein the weak acid cation exchange resin is a hydrogen form of a weak acid cation exchange resin and comprises carboxylic acid functional groups and the strong acid cation exchange resin is a hydrogen form of a strong acid cation exchange resin and comprises sulfonic acid functional groups.

15. The kit according to any one of claims 1-14, wherein the kit further comprises d) a premeasured amount of a surfactant.

16. The kit according to any one of claims 1-15, wherein component a), a mixture of weak acid cation exchange resin and strong cation exchange resin component b), and optionally component d), are each packaged in an individual, air-tight and water-tight container.

17. The kit according to any one of claims 1-15, wherein component a), the weak acid cation exchange resin of component b), the strong cation exchange resin component of component b), and optionally component d), are each packaged in an individual, air-tight and water-tight container.

18. The kit according to claim 17, wherein the premeasured amount of the surfactant is present and is present in an amount necessary to constitute 0.1% to 5% by weight of the hypochlorous acid solution once prepared.

19. The kit according to claim 18, wherein the premeasured amount of the surfactant is an amount necessary to constitute 0.5% to 1% by weight of the hypochlorous acid solution once prepared.

20. The kit according to any of claims 15-19, wherein the surfactant is selected from the group consisting of dioctyl sulfosuccinates, alkyldiphenyloxide disulfonates, polysorbates, ethoxylated polyoxypropylene block copolymer, alcohol ethoxylates, sodium polyacrylate polymer, sodium lauriminodipropionate, sodium octyliminodipropionate, and combinations thereof.

21. The kit according to any one of claim 16 or 17, wherein the individual, air-tight and water-tight containers are 3-ply bags comprising oriented polypropylene, metalized polyethylene terephthalate, and linear low-density polyethylene.

22. The kit according to claim any one of claims 1-21, wherein the instructions for preparing a hypochlorous acid solution provide for admixing component a), component b), optionally component d), and a predetermined amount of water.

23. A process for producing hypochlorous acid, the process comprising dissolving a premeasured amount of calcium hypochlorite in water, adding a premeasured amount of a mixture of a weak acid cation exchange resin and a strong acid cation exchange resin to water, and stirring, wherein the premeasured amounts of the calcium hypochlorite and the mixture and water are in effective amounts to produce a hypochlorous acid solution which has a pH of about 4 to about 7 when stirring is halted.

24. The process according to claim 23, wherein the free available chlorine concentration of the hypochlorous acid solution is between about 7000 ppm and about 50 ppm

25. The process according to claim 23, wherein the free available chlorine concentration of the hypochlorous acid solution is between about 1000 ppm and about 200 ppm.

26. The process according to claim 25, wherein the free available chlorine concentration of the hypochlorous acid solution is between about 1000 ppm and about 700 ppm

27. The process according to any one of claim 23, wherein the free available chlorine concentration of the hypochlorous acid solution is between about 200 ppm and about 50 ppm.

28. The process according to claim 26, wherein the free available chlorine concentration of the hypochlorous acid solution remains between about 1000 and 700 ppm when stored at room temperature for up to 48 hours.

29. The process according to claim 27, wherein the free available chlorine concentration of the hypochlorous acid solution remains between about 200 and 50 ppm when stored at room temperature for up to 48 hours.

30. The process according to claim 26, wherein the premeasured amount of calcium hypochlorite is in an amount of about 1.7 g/L to about 1.8 g/L for a predetermined amount of water.

31. The process according to claim 30, wherein the premeasured mixture of the weak acid cation exchange resin and the strong acid cation exchange resin is provided in an amount to provide 6 g/L to about 20 g/L of the weak acid cation exchange resin for a predetermined amount of water; and about 6 g/L to about 12 g/L of a strong acid cation exchange resin for a predetermined amount of water.

32. The process according to claim 31, wherein the premeasured mixture of the weak acid cation exchange resin and the strong acid cation exchange resin is provided in an amount equal to 10 g of weak acid cation exchange resin/L of water based upon a predetermined amount of water; and about 8 g of the strong acid cation exchange resin/L based upon a predetermined amount of water.

33. The process according to claim 27, wherein the premeasured amount of calcium hypochlorite is in an amount of about 0.1 g/L to about 0.2 g/L for a predetermined amount of water.

34. The process according to claim 33, wherein the premeasured mixture of the weak acid cation exchange resin and the strong acid cation exchange resin is provided in an amount to provide 0.3 g/L to about 2.3 g/L of the weak acid cation exchange resin for a predetermined amount of water; and about 0.3 g/L to about 1.4 g/L of a strong acid cation exchange resin for a predetermined amount of water.

35. The process according to claim 34, wherein the premeasured mixture of the weak acid cation exchange resin and the strong acid cation exchange resin is provided in an amount equal to about 1.1 g of weak acid cation exchange resin/L of water based upon a predetermined amount of water; and about 0.9 g of the strong acid cation exchange resin/L based upon a predetermined amount of water.

36. The process according to any one of claims 23-35, the process further comprising adding a premeasured amount of a surfactant with stirring.

37. The process according to claim 36, wherein the premeasured amount of the surfactant is an amount necessary to constitute 0.1% to 5% by weight of the hypochlorous acid solution once prepared.

38. The process according to claim 37, wherein the premeasured amount of the surfactant is an amount necessary to constitute 0.5% to 1% by weight of the hypochlorous acid solution once prepared.

39. The process according to any of claims 36-38, wherein the surfactant is selected from the group consisting of dioctyl sulfosuccinates, alkyldiphenyloxide disulfonates, polysorbates, ethoxylated polyoxypropylene block copolymer, alcohol ethoxylates, sodium polyacrylate polymer, sodium lauriminodipropionate, sodium octyliminodipropionate, and combinations thereof.

40. The process according to any one of claims 23-39, wherein the solution is stirred for about 3 minutes to about 5 minutes.

41. The process according to claim 40, wherein the solution is stirred for about 4 minutes.

42. The process according to any one of claims 23-41, wherein the weak acid cation exchange resin is a hydrogen form of a weak acid cation exchange resin and comprises carboxylic acid functional groups and the strong acid cation exchange resin is a hydrogen form of a strong acid cation exchange resin and comprises sulfonic acid functional groups.

43. The process according to any one of claims 23-42, wherein the calcium hypochlorite, the weak acid cation exchange resin, the strong acid cation exchange resin, and optionally a surfactant, are admixed by pouring from individual packages for a) calcium hypochlorite and b) the mixture of weak acid cation exchange resin and strong acid cation exchange resin; or admixed by pouring from individual packages for a) calcium hypochlorite, and b1) the weak acid cation exchange resin, and b2) the strong acid cation exchange resin.

44. The process according to claim 43, wherein the calcium hypochlorite is packaged in a first 3-ply bag, the mixture of weak acid cation exchange resin and strong acid cation exchange resin is packaged in a second 3-ply bag, and optionally a surfactant is packaged in a third 3-ply bag, and the calcium hypochlorite and mixture of resins, and optionally the surfactant, are admixed with a predetermined amount of water.

45. The process according to claim 43, wherein the calcium hypochlorite is packaged in a first 3-ply bag, the weak acid cation exchange resin is packaged in a second 3-ply bag, the strong acid cation exchange resin is packaged in a third 3-ply bag, and optionally a surfactant is packaged in a fourth 3-ply bag, and the calcium hypochlorite and the cation exchange resins, and optionally the surfactant, are admixed with a predetermined amount of water.

46. The process according to claim 23, wherein the hypochlorous acid solution is further diluted with water.

47. The kit according to claim 1, wherein the premeasured amounts of calcium hypochlorite and mixture of weak acid cation exchange resin and strong acid cation exchange resin are present in amounts effective to produce hypochlorite acid in an amount sufficient to reduce the concentration of a measured microorganism by 99% or more when remaining in contact with the surface for about 30 seconds to about 2 minutes.

48. The process according to claim 23, wherein the premeasured amounts of calcium hypochlorite and mixture of weak acid cation exchange resin and strong acid cation exchange resin are present in amounts effective to produce hypochlorite acid in an amount sufficient to reduce the concentration of a measured microorganism by 99% or more when remaining in contact with the surface for about 30 seconds to about 2 minutes.
